(19) United States
(12) Patent Application Publication
(10) Pub. No.: US 2012/0101057 A1
(43) Pub. Date: Apr. 26, 2012

(54) COMBINED THERAPY OF COLORECTAL CARCINOMA

(75) Inventors: Joanna Wietrzyk, Wroclaw (PL); Magdalena Milczarek, Wroclaw (PL); Andrzej Kutner, Warszawa (PL); Michal Chodynski, Pruszkow (PL)

(73) Assignee: INSTYTUT FARMACEUTYCZNY, WARSZAWA (PL)

(21) Appl. No.: 13/375,653

(22) PCT Filed: Jun. 9, 2010
(86) PCT No.: PCT/PL10/00044

§ 371 Date: Dec. 21, 2011

(30) Foreign Application Priority Data
Jun. 10, 2009 (PL) P-388252

(51) Int. Cl. A61K 31/7068; A61P 35/04; A61P 35/00; A61K 31/59; A61K 31/593

(52) U.S. Cl. 514/49; 514/167

(57) ABSTRACT

The invention relates to a combined therapy, wherein addition of a vitamin D analogue to the standard treatment regimen based on cytostatics, 5-fluorouracil and/or its precursors, generates potential possibility of achieving beneficial therapeutic effect in the first-line chemotherapy or adjuvant therapy of colorectal carcinoma. Vitamin D analogues are selected from a group consisting of tacalcitol, calcipotriol and 5,6-trans-isomer of calcipotriol.

### FIELD OF THE INVENTION

The invention relates to a combined therapy of colorectal carcinoma.

Specifically, the invention relates to the treatment wherein an addition of vitamin D analogue to standard regimen based on cytostatic, especially 5-fluorouracil and/or its precursor, generates potential possibilities to achieve a beneficial therapeutic effect in a first-line or adjuvant chemotherapy of colorectal carcinoma.

### BACKGROUND OF THE INVENTION

Colorectal carcinoma consists of about 9.4% of all malignant cancers in men and 10% in women and is the second cause of morbidity due to malignant cancers. In the Central Europe, about 50-70% of colorectal cancers are located in the terminal part of it—i.e., rectum (about 30-50%) and in sigmoid (about 15-20%). This cancer is characterised by a malignant course, metastases are formed mainly in the liver and lungs. Genetic and environmental factors are a base of the colorectal carcinoma. Most sporadic colorectal carcinomas develop as a result of overlapping mutations of suppressor genes APC, DCC and p53, which lead to hyperplasia of glandular epithelium and formation of adenoma, and then to a malignant transformation, as a result of inactivation of oncogens K-ras. Also, numerous cases of hereditary carcinoma are observed which are not related to polyposis. Also, an epigenetic mechanism leading to a functional exclusion of genes and hypermethylation of DNA in promoter regions of genes hMLH1, APC and p16, plays a significant role. The probability of occurrence of colorectal carcinoma is increased by environmental factors, such as the presence of carcinogenic factors in the consumed food, smoking or inappropriate diet and internal factors, for example ulcerative colitis, Leśniowski-Crohn disease, Lynch syndrome, Muir-Torre syndrome, Gardner syndrome, Turcot syndrome, past diseases and a family history. A radical therapy of colorectal carcinoma consists in removal of diseased part of the intestine together with a region of confluence of lymph. The standard treatment is chemotherapy. The introduction of cytostatics to clinical practice, such as 5-fluorouracil, irinotecan, oxaliplatin al lowed a significant prolongation of survival in the course of this neoplasm. However, despite the advance of medicine, the results of treatment are still not satisfactory. 5-year survivals reach 20%. The reason of this phenomenon is thought to be, first of all, the late diagnosis of the disease [“Onkologia” edited by A. Kulakowski and A. Skowrońska-Gardas, PZWL 2003].

The most commonly used anticancer chemotherapeutic in the treatment of colorectal carcinoma is 5-fluorouracil (5-FU). 5-FU is classified as antimetabolite and it has a complex mechanism of action. After penetrating into a cell, it is transformed, with participation of fosforylase and thymidine kinase, to 5-fluoro-2′-deoxyurydine monophosphate (5-FdUMP), which is an active metabolite. 5-FdUMP forms a complex with thymidine synthetase, which leads to inhibition of its activity and, as a consequence, to inhibition of DNA synthesis. Moreover, 5-FU simultaneously undergoes metabolism in two different biochemical pathways, which lead to formation of 5-fluoro-2′-deoxyuridine triphosphate (5-FdUTP) and 5-fluorouridine triphosphate (5-FUTP). 5-FdUTP is built in as a substrate to DNA during its synthesis. This abnormality is readily removed by uracil glycosylase and, as a result, DNA looses integrity by fragmentation. However, 5-FUTP metabolite is built in newly formed RNA molecules, which leads to an inhibition of protein synthesis. 5-FU, by disturbing of DNA and RNA synthesis, leads to disturbance of growth, damage and death of cells. 5-FU is given to colorectal carcinoma patients in combination with leucovorin, which stabilizes binding of 5-FU with thymidyne synthetase, what facilitates inhibition of DNA synthesis.

Precursor of 5-fluorouracil-fluoropyrimidine carbaminate, known under a generic name of capecitabine, is deprived of its cytotoxic properties, only as a result of activation after sequential, enzymatic biotransformation to 5-FU. Orally administered capecitabine is absorbed from the gastrointestinal tract in unchanged form. The first of the three transformations occurs in the liver with participation of carboxyestherase to 5-deoksy-5-fluorocitidine (5′-DFC). This compound is then transformed to 5-deoxy-5-fluorouridine (5′-DFUR) by citidine deaminase, which is found mainly in the liver and in the neoplastic tissue. The activity of the enzyme responsible for a final conversion to 5-FU-thymidine phosphorylase (ThyPase), is 4-fold greater in the tissue of primary tumours of colorectal carcinoma in relation to surrounding normal tissues. The results of immunohistochemical investigations indicate cells of neoplasm stroma as a main localisation of thymidine phosphorylase. Metabolism of 5-FU in anabolic pathway leads to blockade of methylation of deoxyuridil acid to thymidil acid, which results in influence on deoxyribonuclein acid (DNA). Incorporation of 5-FU leads also to an inhibition of RNA and protein synthesis. Due to significant importance of DNA and RNA for cell division and growth, thymidine deficiency caused by 5-FU may lead to growth disturbances and cell death. Effects of disturbances of DNA and RNA synthesis are greatest in cells which undergo fast division, which readily metabolise 5-FU.

A new direction in colorectal cancer treatment is a targeted therapy and combining drugs of different mechanism of action. Despite progress in diagnostics and treatment of colorectal cancer, there is still need for new, more efficient therapies which may be an advance in the therapy of this disease.

Active metabolites and cholecalciferol (vitamin D) analogues influence the calcium-phosphate balance and are used in treatment of metabolic diseases and skeleton system disease, especially osteoporosis. Moreover, the main natural active form of vitamin D, 1α,25-dihydroxycholecalciferol (calcitriol) and its synthetic analogues show antiproliferative action and beneficially influence differentiation of cancer cells and epidermal keratinocytes. Due to this, cholecalciferol analogues are used in treatment of hyperproliferative diseases, especially skin diseases such as some forms of plaque psoriasis, ichtiosis and keratosis (Beckman M. J., DeLuca H. F., Modern view of vitamin D₃ and its medicinal uses, w: Ellis G P, Luscombe D K, Oxford A W, ed., Progress in Medicinal Chemistry. t.35, Amsterdam, Elsevier Science Publishers, 1998, p. 1-56; Vitamin D, t. I, ed. II, edited by D. Feldman, Amsterdam, Elsevier Science Publishers, 2005).

Among cholecalciferol analogues 1,24(R)-dihydroxycholekalciferol is of special interest, this is a precursor of 1,24(R),25-trihydroxycholecalciferol, a natural calcitriol metabolite of generic name tacalcitol (Peters D. C., Balfour J. A., Tacalcitol., Drugs 1997; 54:265-271). Contrary to other drugs of vitamin D analogues group, tacalcitol does not show calcium side effects, therefore it may be administered in other diseases in higher doses than in dermatology and moreover, it qualifies to general administration.

It was demonstrated in the description of Polish patent application P-339112 that tacalcitol showed also a beneficial effect in vitro and in vivo on differentiation and inhibition of proliferation of some lines of human cancer cells. At the same time, activity of tacalcitol is statistically significantly higher than activity of its C-24 diastereomer (1α,24(S)-dihydroxycholecalciferol) and natural calcitriol.

Calcipotriol, (1S,3R,5Z,7E,22E,24S)-24-cyclopropyl-9,10-secochola-5,7,10(19),22-tetraeno-1,3,24-triol (M. J. Calverley, Tetrahedron 43, 4609 (1987)), also exerts a beneficial influence on inhibition of excessive proliferation of epidermal keratinocites (F. A. C. M. Castelijins i wsp. Acta Derm. Venereol. 79, 111 (1999)). Studies carried out in rats showed that it exerts 100-200-fold lesser influence on calcium metabolism than calcitriol (L. Binderup, E. Bramm, Biochem. Pharmacol. 37, 889 (1988)).

In the Polish patent application P-378586, use of structural analogues of calcipotriol for preparation of pharmaceutical of pharmaceutical agents of antineoplastic activity was claimed. The above mentioned analogues are selected from a group consisting of (1S,3R,5Z,7E,22E,24R)-24-cyclopropyl-9,10-secochol-5,7,10(19),22-tetraeno-1,3,24-triol ((24R)-isomer), (1S,3R,5Z,7E,22Z,24S)-24-cyclopropyl-9,10-secochol-5,7,10(19),22-tetraeno-1,3,24-triol (22,23-cis isomer), (1R,3R,5Z,7E,22E,24S)-24-cyclopropyl-9,10-secochol-5,7,10(19),22-tetraen-1,3,24-triol (1β-0H-isomer) and (1S,3R,5E,7E,22E,24S)-24-cyclopropyl-9,10-secochol-5,7,10(19),22-tetraeno-1,3,24-triol (5,6-trans-isomer).

At present, it was shown in the studies in vitro on selected cell lines of human colorectal carcinoma, confirmed in animals models, that vitamin D analogues used in combination with some cytostatics show synergistic antineoplastic action.

### SUMMARY OF THE INVENTION

The present invention provides the use of vitamin D analogue with a cytostatic, 5-fluorouracil and/or its precursor in the combined therapy of colorectal carcinoma.

The present invention is based on the finding that administration of 5-FU or its precursor in combination with some vitamin D analogues in vivo results in enhancement of antineoplastic effect in comparison with the use of 5-FU or its precursor only. It has been shown that vitamin D analogues, especially tacalcitol and 5,6-trans-isomer of calcipotriol, show similar efficacy in inhibiting tumour growth in combined therapy. In addition, in case of 5,6-trans-isomer of calcipotriol, a beneficial effect on prolongation of survival time of mice is observed, while in case of tacalcitol—an anti-metastatic effect of combined therapy.

Beneficial combined effect of 5-FU with vitamin D analogues is observed in long time periods, presumably due to mechanism of action of the latter—their differentiating influence on neoplastic cells which is seen after appropriately long time of incubation (J. Wietrzyk et al.: Toxicity and antineoplastic effect of (24R)-1,24-dihydroxyvitamin D₃ (PRI-2191). Steroids, 69/10: 629-635, 2004; A. Opolski i wsp.: Biological activity in vitro of side-chain modified analogues of calcitriol. Current Pharmaceutical Design, 6: 755-765, 2000.)

A beneficial prognostic factor (M. G. Anderson et al.: Expression of VDR and CYP24A1 mRNA in human tumors. Cancer Chemother. Pharmacol. 57, 234-240, 2006), indicating possible benefits from the use of therapeutic regimen containing vitamin D analogue and 5-FU, is an expression of vitamin D receptor, increasing both after use of 5-FU, and vitamin D analogues in combination with 5-FU.

It was found at the same time that toxicity of combined therapy, measured by such parameters as body mass of mice and calcium level in plasma, is not a limiting factor of potential possibility of use of antineoplastic combination according to the invention in humans. A body mass of mice treated with 5-FU in combination with vitamin D analogue does not differ significantly from a body mass of mice treated with 5-FU only. In some cases, an increased loss of body mass of mice treated with 5-FU in combination with tacalcitol is observed that is related to significantly higher calcium activity in comparison with 5,6-trans-isomer of calcipotriol, but at the same time, calcium activity and toxicity of tacalcitol is much lower than that of calcitriol (J. Wietrzyk et al.: The antitumor effect of lowered doses of cytostatics combined with new analogs of vitamin D in mice. Anticancer Res., 27: 3387-98, 2007; J. Wietrzyk et al.: Toxicity and antineoplastic effect of (24R)-1,24-dihydroxyvitamin D₃ (PRI-2191). Steroids, 69/10: 629-635, 2004.).

In view of the obtained results, in a preferred embodiment of the present invention vitamin D analogues are selected from a group consisting of tacalcitol, calcipotriol and 5,6-trans-isomer of calcipotriol.

The components of the combination may be administered to the patient as a pharmaceutical formulation in a fixed dosage form comprising the therapeutically effective amounts of the active substances in combination with pharmaceutically acceptable carriers and/or excipients. Alternatively, the components of the combination may be administered to the patient in individual unite dosage forms, that may be given either simultaneously or sequentially in order and time intervals predetermined by the clinician or medical practitioner.

Pharmaceutical preparations, beside the active substance, may contain known pharmaceutically acceptable carriers and/or excipients appropriate for a given pharmaceutical form, not having their own pharmacological action and adverse reactions with the active substance.

Therapeutically effective dose of the active substance in treatment of colorectal cancer may be established by a clinician based on clinical trials and adjusted to the medical condition, age and body weight of the patient, as well as the route of administration and individual response to therapy.

The effective daily dose of vitamin D analogue in case of an adult human may be 0.1 do 200 μg, preferably from 0.5 to 50 μg. Daily dose may be administered to the patient as single unit dose once daily or divided into several daily doses in determined time intervals.

5-Fluorouracil or its prodrug capecitabine may be administered orally according to currently accepted standard regimens of colorectal cancers treatment. In case of capecitabine, the initial dose is 2.500 mg/m²/daily taken twice daily, administered in cycles including 2 weeks of everyday treatment followed by one week without taking the medication. The dose may be decreased by attending physician with regard to intensity of therapeutic action of cytostatic as a result of simultaneous administration of vitamin D analogue according to the present invention.

The components of the combination of vitamin D analogue with cytostatic according to the invention may be formulated in the pharmaceutical form acceptable for systematic administration, for example orally, such as tablets, capsules, film-coated tablets, enteric coated tablets; in the form acceptable for parenteral use, such as solutions, suspensions or lyophilisate for reconstitution ex tempore; or in the form for local administration. The selection and amount of carriers and excipients depends on the form and route of administration of the agent. The appropriate drug form may be formulated with use of techniques well known to those skilled in the art, using any pharmaceutically carriers, solvents, fillers and other excipients.

Especially convenient route of administration of combination according to the invention is an oral route.

Therefore, the preferred component of a combination according to the invention is capecitabine which, contrary to 5-FU, may be administered orally and reaches a higher concentrations in neoplastic cells.

A pharmaceutical preparation for administration of vitamin D analogues by oral route may especially be in the form of capsules. In this case, the active substance is combined with a carrier and gelatin capsules are filled with the obtained composition. Capsule filling is in the form of oil solution, suspension or emulsion. Appropriate carriers include, for example castor, coconut, olive, palm, corn, peanut oil, synthetic and natural triglycerides of fatty acids, unsaturated medium-chain fatty acids, modified long-chain fatty acids, glycol esters, polyethylene glycols and others. Appropriate excipients are tensides, for example lecithine, mono- and diglycerides and esters of polyoxyethylenesorbitan.

Capsules may be soft and hard gelatin capsules, differing by composition of gelatin shell for its preparation. Gelatin shell in case of soft capsules include plastisizers, such as glycerol, sorbitol; preservatives, such as benzoic acid and its salts, alkyl hydroxybenzoates; colourants and flavourings.

Pharmaceutical formulation for parenteral administration may be in the form of suspension ready to use, lyophilisate form for reconstitution ex tempore or a concentrate for preparation of intravenous infusions. Carriers appropriate for intravenous pharmaceutical formulations include, for example, sterile aqueous solutions, such as saline solution, carbohydrate solution, for example glucose, mannitol, dextrose lactose and aqueous solutions of buffers, for example phosphate buffer. Moreover, the agent may contain other excipients, conventionally used in order to ensure osmolarity, antioxidants, preservatives and others.

The combination according to the invention may be used in treatment of colon cancer, metastatic colorectal cancer and in supportive treatment after surgery of colorectal cancer.

Possibility of use of combination components in oral form, either in a fixed-dose formulation or in separate formulations allows to modify an effective dose during treatment, that is very important in some clinical situations. Moreover, oral route of administration is convenient for patients and allows to maintain good quality of life.

### Biological Activity

In a further description, the following markings of vitamin D analogues are used:
PRI-2191—tacalcitol;
PRI-2201—calcipotriol;
PRI-2205—5,6-trans-isomer of calcipotriol.
In the studies in vitro, calcitriol (1,25-dihydroxycholekalciferol) was used, as naturally occurring biologically active form of vitamin D.

I. In Vitro Studies

1. The Influence of Calcitriol and Vitamin D Analogues In Vitro on Antiproliferative Activity of 5-Fluorouracil in Human and Mouse Colorectal Carcinomas.

The influence of calcitriol and vitamin D analogues: PRI-2191, PRI-2201, PRI-2205 on antiproliferative activity of 5-fluorouracil was studied in vitro.

After 24 h culture, the cells of large intestine carcinoma were treated simultaneously with calcitriol or vitamin D analogues and 5-fluorouracil for 120 hours. The final concentration of vitamin D analogues was 100 nM, and of 5-fluorouracil—-1 μg/ml or 0.1 μg/ml. Statistical analysis of the obtained results of inhibition of proliferation was carried out by analysis of percent of inhibition of proliferation in individual combinations of compounds.

Hypothetical value was calculated according to a formula:

% H=100−[(100−% cyt)*(100−% vit)]/100

wherein:
% H—hypothetical inhibition of proliferation by combination of compounds [Vo];
% cyt—inhibition of proliferation by 5-Fu alone [%];
% vit—inhibition of proliferation by calcitriol or cholecalciferol analogue alone [%];

The obtained results were assigned the following values:

− antagonistic effect,

+ sub-additive effect

++ additive effect,

+++ synergistic effect.

The hypothetical values below experimental value determined for combination of compounds prove the synergism of action of the compounds. Additive effect occurs when the experimental and hypothetical value are similar, sub-additive effect occurs when experimental value is lower than hypothetical value but above value for a cytostatic alone, and antagonistic action—when a value determined for a cytostatic used alone is above experimental value for combination of both compounds. [Peters G. J. i wsp.: Pharmacol Ther 2000, 87, 227-253]

TABLE 1.1
Type of reaction between vitamin D analogues and 5-fluorouracil
after 120-hours exposure of large intestine carcinoma
cells to the tested compounds.

| Vitamin D | 5-FLUOROURACIL | | | | |
|---|---|---|---|---|---|
| analogue | 0.1 μg/ml | 0.1 μg/ml | 0.01 μg/ml | 1 μg/ml | 1 μg/ml |
| [100 nM] | HT-29 | MC38/0 | | CaCo-2 | LoVo |
| CALCITRIOL | +++ | +++ | +++ | − | +++ |
| PRI-2191 | +++ | +++ | +++ | +++ | +++ |
| PRI-2201 | +++ | +++ | +++ | +++ | +++ |
| PRI-2205 | +++ | +++ | +++ | +++ | +++ |

+ sub-additive effect;
++ additive effect;
+++ synergistic effect:
− antagonism

As a result of 120-hour exposure to the tested compounds in all models of colorectal cancer, synergism in interaction of vitamin D derivatives and 5-fluorouracil was shown, and onfy in one case (cafcitriof Cowards Caco-2 ceil line) antagonism was observed (Table 1.1, FIG. 1.1).

II. In Vivo Studies

1a. Influence of PRI-2191 and PRI-2205 on anticancer activity of 5-fluorouracil (5-FU) in a model of murine colorectal cancer C38
Mice of C57BL/6 strain (female) with colorectal cancer C38, cancer cells implanted subcutaneously 0.25 ml/mouse, cell suspension 33%.
The number of mice in groups: control−8; PRI-2191−8; PRI-2205−8; 5-FU−8; 5-FU +PRI-2191−8; 5-FU+PRI-2205−8.
Treatment of mice was started on 9 day of experiment.
5-FU: dose 150 mg/kg/day, intraperitoneal administration, three times (on days 9, 16, 23).
PRI-2191: dose 2 ug/kg/day, administered subcutaneously per half of body mass, multiple administration, 3 times a week (on days 11, 14, 16, 18, 21, 23).
PRI-2205: dose 10 μg/kg/day, administered subcutaneously per half of body multiple administration, 3 times a week (on days 11, 14, 16, 18, 21, 23, 25, 28).
Measurements of subcutaneous tumours were carried out during experiment.

TABLE 2.1
Influence of vitamin D analogues on tumour growth in mice with
colorectal cancer C38 treated with 5-fluorouracil.

| | Tumours-28 DAY OF EXPERIMENT | | | |
|---|---|---|---|---|
| GROUPS | MEAN ± SD [g] | TGI [%] | TGI hip [%] | N-28 DAY |
| CONTROL | 1.23 ± 0.72 | − | − | 5 |
| PRI-2191 | 0.88 ± 0.49 | 16 | − | 4 |
| PRI-2205 | 1.06 ± 0.84 | 29 | − | 7 |
| 5-FU | 0.19 ± 0.19* | 84 | − | 8 |
| 5-FU + PRI-2191 | 0.076 ± 0.076* | 94 | 87 | 6 |
| 5-FU + PRI-2205 | 0.043 ± 0.043* | 98 | 89 | 7 |

*p < 0.05 Mann-Whitney U test as comparison with control
N—number of mice in a group,

5-FU decreases in statistically significant manner volume of tumours C38 in comparison with control starting from day 8 in all days of measurement; it is similar in case of combination of 5-FU with analogues of vitamin D. PRI-2191 inhibits 38C tumours growth in the last four measurements (day 25, 28, 30 and 32). PRI-2205 inhibits C38 tumours growth from 18 to 28 day of measurement The use of combination of both PRI-2191, and PRI-2205 with 5-FU decreases in statistically significant manner volume of tumours also in comparison with 5-FU (FIG. 2.1}.

Mathematical TGI analysis (Tumor Growth Inhibition) by means of formulas allowing determination of type of reaction of the tested compound combinations indicates that in all days of measurement, the hypothetical TGI values are lower than value obtained experimentally. The obtained result indicates a synergism in reaction of these compounds in inhibition of tumours of murine colorectal carcinoma C38.

1.b The influence of PRI-2205 PRI-2191 on anticancer activity of 5-fluorouracil (5-FU) in a model of murine colorectal cancer C38—the evaluation of survival time of mice.

Mice with colorectal cancer C38 were treated with 5-FU given in a single administration, intraperitoneally on day 1 at a dose of 75 mg/kg. PRI-2191 and PRI-2205 were given 3 times per week at a dose 2 or 10 μg/kg/day (10 times, on days: 1, 3, 6, 8, 10, 13, 15, 17, 20, 22) Measurements of subcutaneous tumours were carried out during experiment, moreover survival time of animals with tumour was evaluated.

Statistical analysis of the obtained results suggests that PRI-2191 has no influence on survival time of mice treated with 5-FU, whereas PRI-2205 prolongs survival of mice treated with 5-FU (p<0.05) (FIG. 6).

5-FU decreases in a statistically significant manner C38 tumour mass in comparison with control starting from day 8 in all days of measurement The results are similar in case of combination of 5-FU with vitamin D analogues. However. PRI-2191 alone inhibits C38 tumour growth only in two last measurements (day 17 and 20). PRI-2205 does not influence tumour mass. The use of combination of both PRI-2191 and PRI-2205 with 5-FU in a statistically significant manner decreases tumour mass also in comparison with 5-FU (example in FIG. 7).

Mathematical analysis of ILS parameters (Increase in Life Span) and TGI (Tumor Growth Inhibition) proves that in all days of measurement, hypothetical TGI values are significantly lower than the value obtained experimentally, what indicates a synergism of action of these compounds in inhibition of tumour growth of the murine colorectal carcinoma C38 (Table 2). A synergistic efFect of action on survival time was found only in case of combination of PRI- 2205 analogue with 5-FU (Table 2).

TABLE 2.2
Prolongation of survival time and inhibition of C38 tumour growth under
influence of the treatment (experimental and hypothetical values)

| | ILS | | | TGI (d20) | | |
|---|---|---|---|---|---|---|
| Compounds | % | % H | Effect | % | % H | Effect |
| 5-FU | 21 | | | 64 | | |
| PRI-2191 | 52 | | | 44 | | |
| PRI-2205 | 2 | | | 0 | | |
| PRI-2191 + 5-FU | 41 | 62 | antagonism | 90 | 80 | synergism |
| PRI-2205 + 5-FU | 77 | 23 | synergism | 88 | 64 | synergism |

% H = 100-[(100-TGI CY) × (100-TGI calcitriol analogue)/100]

1.c. Influence of PRI-2205 and PRI-2191 on anticancer activity of 5-fluorouracil (5-FU) in a model of murine colorectal cancer C38 - evaluation of a burden of metastases in regional lymph nodes.

Cancer cells of murine colorectal cancer MC38/EGFP (cells expressing a protein of green fluorescence) were implanted subcutaneously in an amount of 1×106 cells/mouse. Treatment started on day 19, 5-FU was given in a concentration of 75 mg/kg i.p. every 5 days (on days 19, 24, 29) and anlogues of vitamin D: PRI-2191 was given in concentration of 1 μg/kg or PRI-2205 in concentration of 10 μg/kg. Compounds were given subcutaneously, 3 times a week. On day 50 of experiment, axillary and inguinal lymph nodes were taken for analysis from mice of three groups: CONTROL, PRI-2191 and PRI-2205. Then on day 59 of experiment, lymph nodes from mice of remaining were taken for analysis. Using NightOWL II LB 983 apparatus, photographs of isolated lymph nodes were taken in order to visualize cancer cells in sites of metastases.

Intensity of the measured fluorescence is related to a burden of metastases (the number of cells with fluorescence). The observed metastases in regional lymph nodes of control mice treated with PRI-2191 or PRI-2205 only did not differ significantly (data not shown), however while evaluating intensity of fluorescence of lymph nodes taken from mice treated 5-FU together with PRI-2191, a statistically significant difference was shown which indicates an increased anti-metastatic action, occurring in case of combined treatment with 5-FU and PRI-2191 (FIG. 2.6).

PRI-2205 did not influence the metastatic process of this carcinoma.

TABLE 2.3
Influence of vitamin D analogues on tumour growth in mice with
colorectal cancer HT-29 treated with 5-fluorouracil.

| | TUMOURS - 49 DAY OF EKSPERIMENT | | | |
|---|---|---|---|---|
| GROUPS | MEAN ± SD [g] | TGI [%] | TGI hip [%] | N-49 DAY |
| CONTROL | 2.02 ± 0.38 | − | − | 7 |
| PRI-2191 | 2.19 ± 1.51 | 16 | − | 4 |
| PRI-2205 | 1.88 ± 0.78 | 31 | − | 7 |
| 5-FU | 1.23 ± 0.30* | 47 | − | 8 |
| 5-FU + PRI-2191 | 0.78 ± 0.22*^{#} | 63 | 56 | 8 |
| 5-FU + PRI-2205 | 0.75 ± 0.16*^{#} | 66 | 63 | 8 |

*p < 0.05 Mann-Whitney U test as comparison with control
^{#}p < 0.05 Mann-Whitney U test as comparison with 5-fluorouracil
N—number of mice In a group,

5-FU decreases in a statistically significant manner HT-29 tumour in comparison with control, similarly as combinations of 5-FU with vitamin D analogues. However, PRI-2191 or PRI-2205 alone did not influence tumour mass.

The use of combination of PRI-2191 or PRI-2205 in this regimen with 5-FU influences significantly also tumour mass in comparison with 5-FU alone—a synergism was observed in inhibition of tumour growth by these compounds used in combination (FIG. 2.9).

3. The influence of PRI-2191 and PRI-2205 on anticancer activity of capecitabine (KCY) in a model of murine colorectal carcinoma C38
Mice of C57BL/6 strain (female) with colorectal cancer C38, cancer cells implanted subcutaneously 0,25 ml/mouse, cell suspension 33%.
Number of mice in groups: control - 8; PRI-2191 −8; PRI-2205 −8; 5-FU−8; 5-FU+PRI-2191−8; 5-FU+PRI-2205−8.
Treatment of mice started on day 4 of the experiment.
KCY: dose 250 mg/kg/day, oral administration, multiple 5 times a week (on days 4, 5, 6, 7, 8, 11, 12, 13, 14,15).
PRI-2191: dose 1 μg/kg/day, given subcutaneously per half of body mass, 3 times a week (on days 4, 6, 8, 11, 13, 15, 18, 20, 22, 25, 27).
PRI-2205: dose 10 μg/kg/day, given subcutaneously per half of body weight, multiple administration, 3 times a week (on days 4, 6, 8, 11, 13, 15, 18, 20, 22, 25, 27).
Measurements of subcutaneous tumours size were carried out during the experiment.

TABLE 2.4
Influence of vitamin D analogues on tumour growth in mice with
murine colorectal carcinoma C38 treated with capecitabine.

| | TUMOURS - 18 DAY OF EXSPERIMENT | | | |
|---|---|---|---|---|
| GROUPS | MEAN ± SD [g] | TGI [%] | TGI hip [%] | N-18 DAY |
| CONTROL | 0.72 ± 0.42 | − | − | 8 |
| PRI-2191 | 0.75 ± 0.32 | − | − | 8 |
| PRI-2205 | 0.81 ± 0.60 | − | − | 8 |
| KCY | 0.41 ± 0.33 | 47 | − | 8 |
| KCY + PRI-2191 | 0.29 ± 0.21* | 64 | 41 | 8 |
| KCY + PRI-2205 | 0.22 ± 0.26* | 89 | 36 | 8 |

*p < 0.05 Mann-Whitney U test as comparison with control
N—number of mice in a group

TABLE 2.5
The influence of vitamin D analogues on growth of tumours in mice with
murine colorectal carcinoma C38 treated with capecitabine.

| | TUMOURS - 29 DAY OF EXPERIMENT | | | |
|---|---|---|---|---|
| GROUPS | MEAN ± SD [g] | TGI [%] | TGI hip [%] | N-29 DAY |
| CONTROL | 2.95 ± 2.29 | − | − | 6 |
| KCY | 1.65 ± 1.39 | 35 | − | 6 |
| KCY + PRI-2191 | 0.82 ± 0.59* | 70 | 41 | 5 |
| KCY + PRI-2205 | 0.92 ± 0.49* | 68 | 36 | 6 |

*p < 0.05 Mann-Whitney U test as comparison with control
N—number of mice in a group,

### Conclusions:

Capecitabine, in the used dose and treatment regimen, decreases tumour C38 mass in comparison with control. It is similar in case of combination of capecitabine with vitamin D analogues, whereas, PRI-2191 or PRI-2205 alone does not influence tumour mass.

The use of PRI-2191 or PRI-2205 in combination with capecitabine influences significantly tumour mass in comparison with capecitabine alone. A synergism was observed in inhibition of tumour growth by PRI-2205 analogue used in combination with, however, in a case of PRI-2191 analogue, a synergistic effect is seen mainly in the last days of the experiment.

III. Expression of vitamin D receptor (VDR)

Lisates of human colorectal carcinoma cells HT-29 cultured in vitro

In case of evaluation of influence of compound combination: vitamin D analogue with anticancer drug on expression of vitamin D receptor, the experiment was carried out on disposable scale pans (Sarstedt, Newton, USA). The cells were suspended in a culture medium in the amount of 300 000 cells/ml of medium and put on a scale pan in a volume of 6 ml/well, then medium containing vitamin D analogue PRI-2205 was put on cells (in a volume of 3 ml/scale pan), final concentration of which was 100 nM. After 24 hours, medium containing 5-fluorouracil was put on cells, final concentration of which was 1 μg/ml. Cells were exposed on compound combination for 24 hours. In order to detect a vitamin D receptor D (VDR), Western Blot method was used.

After incubation of cells with the tested compounds, lyse of the cells with the tested compounds in RIPA buffer (Sigma) was carried out, supplemented with cocktail of protease inhibitors (Sigma). Then, in cell lysates, protein concentration was determined (DC Protein Assay, BIO-RAD). The prepared cell lisates were subjected to protein electrophoresis on polyacrylamide gel in a presence of SDS. Proteins resoluted in gel were transferred to nitrocellulosis (electroblotting). Then, using specific antibody of the first layer (VDR-Rabbit polyclonal IgG, Santa Cruz Biotechnology) vitamin D receptor was determined. Detection of the sought proteins was carried out with the use of chemifluorescence (ECF Western Blotting Kit, Amersham Biosciences). The intensity of fluorescence was measured in a Typhoon 8600 scanner.

2. Lysates from cells taken from tumours of colorectal carcinoma HT-29

Mice with colorectal carcinoma HT-29 were treated with 5-FU given four times at a dose of 75 mg/kg/day (on days 11, 18, 25, 32), intraperitoneally. PRI-2205 was given subcutaneously 3 times a week at a dose of 10 μg/kg/ day (on days 11, 13, 15, 18, 20, 22, 25, 27, 29, 32, 34, 36), respectively. After the end of experiment, the mice were sacrificed and tumours were taken for analysis. Determination of VDR receptor in cell lysates from taken tumours was carried out with the use of Western Blot method described above.

It was observed that PRI-2205 analogue used as a single agent, and in combination with 5-fluorouracil causes an increase of expression of vitamin D receptor. 5-Fluorouracil used as a single agent also induces expression of vitamin D receptor, but to a lesser degree than the used combination of both compounds (FIG. 3.1 and FIG. 3.2).

IV. Calcium level in blood serum of mice treated with 5-FU in combination with w combination with vitamin D analogues.

Mice of BALB/c strain were treated with 5-FU given in a single administration, intraperitoneally on day 1 at a dose of 50 mg/kg. PRI-2191 and PRI-2205 were given 3 times a week at a dose 2 or 10 .μg/kg/day, respectively (10 times, on days: 1, 3, 6, 8, 10, 13, 15, 17, 20, 22). Calcium level was evaluated (Arsezano 3 method, Olympus AU400; Olympus America Inc., Melville, NY, USA) in blood serum of treated and control mice.

PRI-2205 analogue does not influence calcium level in blood serum of mice, however, after administration of PRI-2191, calcium level increases in statistically significant degree (FIG. 4.1).

### Summary:

A beneficial interaction of 5-FU with vitamin D analogues is observed after 120-hour incubation, resulting from mechanism of action of vitamin D analogues—their differentiating influence on cancer cells, which is seen after appropriately long incubation time (J. Wietrzyk et al.: Toxicity and antineoplastic effect of (24R)-1,24-dihydroxyvitamin D₃ (PRI-2191). Steroids, 69/10: 629-635, 2004; A. Opolski et al.: Biological activity in vitro of side-chain modified analogues of calcitriol. Current Pharmaceutical Design, 6: 755-765, 2000.)

2. Combined administration of 5-FU or capecitabine with vitamin D analogues in vivo results in enhancement of anticancer effect in comparison with use of 5-FU alone or capecitabine alone. Both vitamin D analogues: tacalcitol and 5,6-trans-isomer of calcipotriol are similarly efficacious concerning a parameter of inhibition of tumour growth in combined therapy. Additionally, in case of PRI-2205 analogue, a beneficial effect, ie. prolongation of survival time of mice, was observed. Anti-metastatic effect of combined therapy was observed in case of PRI-2191 analogue.
3. The results of examination of expression of vitamin D receptor indicating that its expression increases, both after use of 5-FU, and of analogues in combination with 5-FU prove possible benefits of use of therapeutic regimen containing vitamin D analogue and 5-FU, because high expression of vitamin D receptor is a beneficial prognostic factor (M. G. Anderson et al.: Expression of VDR and CYP24A1 mRNA in human tumors. Cancer Chemother. Pharmacol. 57, 234-240, 2006.)
4. Regarding parameters evaluating potential toxicity of the used therapy (body mass of mice, calcium level in plasma), it was fund that PRI-2205 analogue dose not influence calcium level in mice blood, however, after administration of PRI-2191, calcium level increases in a statistically significant degree (Figunek 4.1). Body mass of mice treated with 5-FU In combination with vitamin D analogues does not differ significantly from body mass of mice treated with 5-FU alone. In some cases, an increased loss of body mass of mice treated with 5-FU in combination with PRI-2191 analogue, what is related to its definitely higher activity in comparison with PRI-2205 (Fig. 3.1). It was shown at the same time that calcium activity and toxicity of PRI-2191 is significantly lower than those of calcitriol U. Wietrzyk et al.: The antitumor effect of lowered doses of cytostatics combined with new analogs of vitamin D in mice. Anticancer Res., 27: 3387-98, 2007; J. Wietrzyk et al.: Toxicity and antineoplastic effect of (24R)-1,24-dihydroxyvitamin D₃ (PRI-2191). Steroids, 69/10: 629-635, 2004.).

The invention is illustrated by the following example.

### EXAMPLE

Soft gelatin capsules containing 0.50 μg of vitamin D analogue. Composition of capsule content:

| | | |
|---|---|---|
| 5,6-trans-isomer of calcipotriol | 0.50 | μg |
| BHA | 0.02240 | mg |
| citric acid | 0.01680 | mg |
| anhydrous ethanol | 1.29560 | mg |
| soy oil | 78.66470 | mg |

The weighed amount of oil (about 80% of the total) was mixed for 15 minutes in nitrogen atmosphere. To the oil, the active substance dissolved in anhydrous ethanol with BHA and citric acid was added. It was stirred for 20 minutes in nitrogen atmosphere to the complete dissolution of ethanol phase in the oil phase, then the remaining part of the oil was added and stirred for 15 minutes.

At the same time, gelatin shell was prepared of the following composition:

| | |
|---|---|
| gelatin | 67.13% |
| glycerol | 24.06% |
| water | 8.78% |
| Aseptine A | 0.01% |
| Cochineal E120 | 0.02% |

The compounds of the Shell were dissolved in hot water, degassed, stirred for 30 minutes at the temperature of 65° C. The whole together with the filling was transferred to capsulating apparatus, where AT the temperature of about 60°, it was dropped to paraffin cooled to about 0° C. The capsules were then rinsed with tetrachloroethyelene and dried for 48 hours at the temperature of about 30°.

Mean mass of a single capsule was 0.1275 g±10%.
Mean mass of filling of the single capsule was about 0.080 g±10%.
Capsules were packed to orange glass jars and closed with polyethylene stoppers.

1-8. (canceled)

9. Method of treating colorectal carcinoma comprising administering to a patient in need of such treatment a pharmaceutically effective amount of a vitamin D analogue in combination with a cytostatic.

10. The method of claim 9, wherein the vitamin D analogue is selected from a group consisting of tacalcitol, calcipotriol, and (1S,3R,5E,7E,22E,24S)-24-cyclopropyl-9,10-secochol-5,7,10(19),22-tetraene-1,3,24-triol.

11. The method of claim 9, wherein the cytostatic is 5-fluorouracil or its precursor.

12. The method of claim 11, wherein the cytostatic is capecitabine.

13. The method of claim 11, wherein the vitamin D analogue and 5-fluorouracil or its precursor are administered to a patient in a fixed dosage form or in individual unit dosage forms comprising therapeutically effective amounts of the active substances.

14. The method of claim 13, wherein the vitamin D analogue and 5-fluorouracil or its precursor are administered to a patient in a fixed dosage form.

15. The method of claim 11, wherein the vitamin D analogue and 5-fluorouracil or its precursor are administered orally.

16. The method of claim 9, wherein the combination of vitamin D analogue and the cytostatic is also useful as first-line chemotherapy of colorectal carcinoma, large intestine and rectum with metastases, or as adjuvant therapy after surgery of colorectal carcinoma.
